(19) United States
(12) Patent Application Publication
(10) Pub. No.: US 2026/0009720 A1
(43) Pub. Date: Jan. 8, 2026

(54) System for Non-destructive Resistance Monitoring

(71) Applicant: Brian K. Beresford, Shepherd, TX (US)

(72) Inventor: Brian K. Beresford, Shepherd, TX (US)

(21) Appl. No.: 18/762,886

(22) Filed: Jul. 3, 2024

(51) Int. Cl. G01N 17/02; G01N 33/2045

(52) U.S. Cl. CPC G01N 17/02; G01N 33/2045

(57) ABSTRACT

A system for non-destructive resistance monitoring is provided. The system comprises one or more pairs of electrodes, including a first electrode for the application of electrical current and a second electrode for the measurement of potential in close proximity to the first electrode. A measuring instrument is coupled to the electrodes, able to receive an electrical signal and collect material resistance data, enabling the determination of a test object's resistance. This setup facilitates the non-destructive evaluation of metallic structures for corrosion or degradation without requiring direct access to the object's surface, offering a significant advantage in maintaining the integrity and longevity of industrial equipment. The system provides for continuous monitoring of a represented pipe segment over time to estimate the deterioration of the pipe system in critical infrastructure.

### BACKGROUND OF THE INVENTION

The present invention relates to the field of corrosion monitoring and detection in industrial equipment. Specifically, the present invention addresses the challenges associated with non-destructive testing methods for identifying and measuring corrosion in metallic structures, such as piping, vessels, and supports within various industries including oil and gas, chemical, petrochemical, and power generation.

Corrosion of metallic plant equipment poses a significant challenge across numerous industries, leading to reduced structural integrity, safety risks, and increased maintenance costs. Traditional methods for detecting corrosion, particularly in areas that are not readily visible or accessible, have been limited by various shortcomings. For instance, ultrasonic testing, while effective for measuring wall thickness, requires direct access to clean surfaces of the equipment, making it impractical for large-scale or continuous monitoring. Similarly, electromagnetic techniques offer potential for non-destructive evaluation but are often constrained by the need for close proximity to the test surface and can be ineffective in complex geometries or for materials with certain properties.

Moreover, existing methods such as these often necessitate the removal of insulation or coatings and cannot easily measure corrosion under supports or within the interiors of equipment without significant disruption to operations. This not only increases the labor and costs associated with testing but also limits the frequency and comprehensiveness of corrosion monitoring.

Resistance testing methods have emerged as a viable alternative, capable of measuring the resistance of materials to detect changes that may indicate corrosion. However, the reliability of such tests has been hampered by the variability and durability of the electrodes used in the measurements. Traditional approaches using temporary or handheld electrodes suffer from inconsistencies due to variations in the electrode-to-surface contact, and the need for frequent recalibration or replacement of electrodes.

The present invention seeks to overcome these limitations by introducing a system for non-destructive resistance monitoring that utilizes permanently installed electrodes in some embodiments. This ensures consistent, accurate measurements over time while reducing the variability associated with electrode degradation or inconsistent contact. By employing corrosion-resistant stainless steel electrodes that can be welded or mechanically attached to the test object, the system offers a durable, reliable solution for continuous corrosion monitoring. The ability to perform measurements without direct access to the test surface and to integrate these measurements into a networked monitoring system represents a significant advancement over existing techniques.

This invention not only addresses the technical challenges of existing corrosion detection methods but also offers a practical solution for industries seeking to enhance the safety, reliability, and longevity of their infrastructure. By providing a means for continuous, accurate monitoring of corrosion, the invention stands to significantly impact the maintenance strategies and operational efficiencies of facilities prone to corrosion-related issues.

In light of the devices disclosed in the known art, it is submitted that the present invention substantially diverges in design elements and methods from the known art and consequently it is clear that there is a need in the art for an improvement for a system for non-destructive resistance monitoring. In this regard the instant invention substantially fulfills these needs.

### SUMMARY OF THE INVENTION

In view of the foregoing disadvantages inherent in the known types of system for non-destructive resistance monitoring now present in the known art, the present invention provides a new system for non-destructive resistance monitoring that comprises one or more pairs of electrodes that includes a first electrode for application of electrical current and a second electrode for measurement of potential in close proximity to the first electrode. A measuring instrument is coupled to the one or more pairs of electrodes and is configured to receive an electrical signal via the electrodes and collect material resistance data. The electrical signals and material resistance data are interpreted to provide information related to the corrosion of the pipe without requiring destructive testing or theoretical modeling.

It is an objective of the present invention to offer a system for non-destructive resistance monitoring which enables the accurate and reliable detection of corrosion within metallic structures without necessitating direct physical access to the equipment's surface. This system utilizes permanently installed electrodes to measure resistance changes over time, providing a continuous assessment of the material's integrity and thereby facilitating early detection of corrosion.

It is an objective of the present invention to provide a system for non-destructive resistance monitoring that reduces the variability and maintenance requirements traditionally associated with corrosion monitoring techniques. By employing corrosion-resistant stainless steel electrodes and a durable connection method, the system provides long-term performance without the need for frequent recalibration or replacement of components.

It is an objective of the present invention to offer a system for non-destructive resistance monitoring capable of integrating with a networked monitoring system for centralized data collection and analysis. This feature allows for the aggregation of resistance data from multiple points, enabling comprehensive corrosion monitoring and the ability to predict and prevent equipment failure through advanced data analysis techniques.

It is an objective of the present invention to provide a system for non-destructive resistance monitoring that is adaptable to a wide range of industrial applications, including but not limited to oil platforms, chemical plants, and power generation facilities. The system's versatility and ease of installation make it suitable for various environments, particularly those where corrosion poses a significant risk to safety and operational efficiency.

It is an objective of the present invention to offer a system for non-destructive resistance monitoring that enhances the safety and longevity of industrial equipment by enabling proactive maintenance strategies. Through continuous and accurate corrosion monitoring, the system assists in scheduling maintenance and repairs before significant degradation occurs, thus avoiding unplanned downtime and extending the usable life of the equipment.

It is therefore an object of the present invention to provide a new and improved system for non-destructive resistance monitoring that has all of the advantages of the known art and none of the disadvantages.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the system for non-destructive resistance monitoring mounted to a pipe segment.

FIG. 2 shows a view of an embodiment of the system for non-destructive resistance monitoring mounted to a pipe system.

FIG. 3 shows a diagram view of the embodiment of the system for non-destructive resistance monitoring.

### DETAILED DESCRIPTION OF THE INVENTION

Reference is made herein to the attached drawings. Like reference numerals are used throughout the drawings to depict like or similar elements of the system for non-destructive resistance monitoring. For the purposes of presenting a brief and clear description of the present invention, the embodiment discussed will be as used for monitoring a pipe within a processing plant. The electrodes are welded directly to the test object, providing a permanent and low-resistance electrical connection. In this way, the system is non-destructive and provides resistance monitoring of the selected pipe. The figures are intended for representative purposes only and should not be considered to be limiting in any respect.

Reference will now be made in detail to the exemplary embodiment(s) of the invention. References to “one embodiment,” “at least one embodiment,” “an embodiment,” “one example,” “an example,” “for example,” and so on indicate that the embodiment(s) or example(s) may include a feature, structure, characteristic, property, element, or limitation but that not every embodiment or example necessarily includes that feature, structure, characteristic, property, element, or limitation. Further, repeated use of the phrase “in an embodiment” does not necessarily refer to the same embodiment. The term “permanent” or “permanently” is defined as intended to remain in place indefinitely or to last for the entire operational life of the test object (such as pipe 2000) without the need for removal or reinstallation. Non-exhaustive examples of permanent installation techniques include welding the electrodes directly to the test object, using durable adhesives that form a long-lasting bond, or other mechanical fastening methods that do not easily allow for the removal or repositioning of the electrodes.

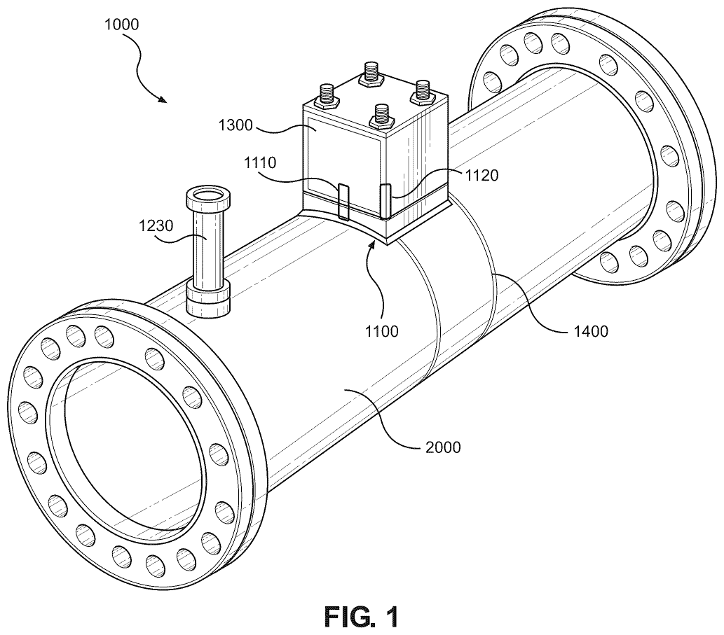

Referring now to FIG. 1, there is a perspective view of an embodiment of the system for non-destructive resistance monitoring mounted to a pipe segment. The system for non-destructive resistance monitoring accurately and reliably detects corrosion within metallic structures, such as piping, vessels, and supports, particularly in industrial settings where such degradation can significantly impact safety, efficiency, and longevity. The system for non-destructive resistance monitoring 1000 comprises one or more pairs of electrodes 1100 configured to introduce controlled electrical current into the metallic structure or test object and measure the potential difference (voltage drop) created by the electrical current flowing through the material from the first electrode. In one embodiment, the one or more pairs of electrodes includes a first pair of electrodes 1110, 1120 and a second pair of electrodes, wherein each pair of electrodes includes a first electrode 1110 for the application of electrical current and a second electrode 1120 for the measurement of potential in close proximity to the first electrode. In the shown embodiment, the first pair of electrodes 1100 are disposed within a housing 1300, wherein the housing 1300 is secured to the pipe via a weld or fastener 1400. A second pairs of electrodes 1100B positioned at a given distance from the first pair of electrodes 1100A. In some embodiments, the distance ranges between 1 meter and 100 meters.

In the shown embodiment, these electrodes 1100 are designed to be permanently installed on the test object (such as a pipe 2000), ensuring consistent and reliable measurements over time. In other embodiments, the system 1000 provides for a modular approach to electrode placement and connection. The electrodes 1100 are integrated into a portable, modular unit that can be temporarily attached to the surface of the test object. Each module comprises a pair of electrodes: one for the application of electrical current 1110 and the other for the measurement of potential 1120, similar to the permanently installed counterparts. However, these electrodes 1100 are housed within a self-contained unit that can be magnetically attached, clamped, or otherwise mechanically secured to the test object without the need for welding or permanent adhesion. In one embodiment, the electrodes 1100 have a pointed tip to facilitate a secure but temporary contact with the test object. This pointed design ensures a consistent electrical connection even in the presence of surface corrosion or coatings.

In the shown embodiment, the electrodes 1100 are made of corrosion-resistant stainless steel, chosen for its durability and resistance to environmental degradation. This material maintains the integrity of the electrical connection and the accuracy of the resistance measurements. The measuring instrument 1200, coupled to the electrodes 1100, is configured to receive an electrical signal transmitted through the electrodes 1100 and to collect material resistance data. This data is indicative of the condition of the test object, with changes in resistance suggesting the presence and progression of corrosion.

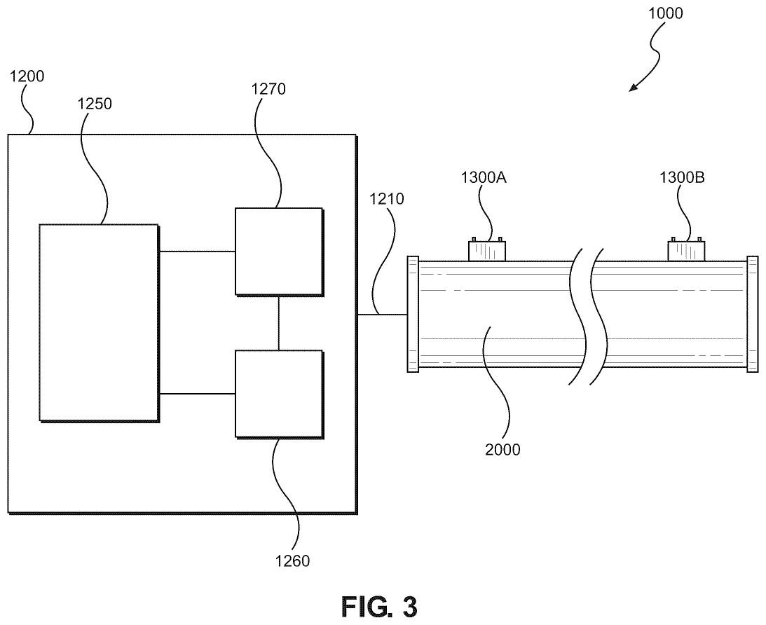

In one embodiment, the system 1000 is configured to integrate with a network (as seen in FIG. 3, 1210), connecting multiple measuring instruments 1200 to a central data collection point. This configuration allows for the aggregation of resistance data from various locations within a facility, enabling comprehensive monitoring and analysis. In a private network configuration, the system 1000 utilizes an intranet or a secure local area network (LAN) to connect the measuring instruments 1200 to the central data collection point. This setup ensures data security and is ideal for sensitive or proprietary operational environments where data confidentiality is paramount. The private network configuration may be employed within a single manufacturing site. The collected data is uploaded to a database, where it is stored and made accessible for further analysis. A processor 1220, connected across the network 1210, is adapted to analyze the plurality of material resistance data, facilitating the detection of corrosion trends and the prediction of equipment degradation.

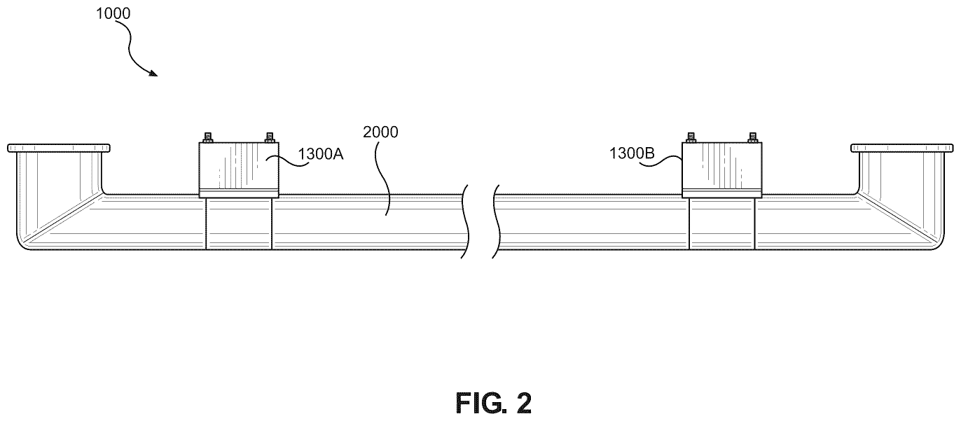

Referring now to FIGS. 1 and 2, there are shown views of an embodiment of the system for non-destructive resistance monitoring 1000 mounted to a pipe system 2000. The system 1000 includes a temperature sensor 1230 for detecting ambient temperature data. The system 1000 is adapted to compensate the resistance measurements for temperature variations, as the electrical resistance of metallic components can change with temperature. By continuously monitoring the temperature and adjusting the resistance data accordingly, the system 1000 ensures the accuracy of corrosion detection under varying environmental conditions.

As shown in FIG. 1, the temperature sensor 1230 is disposed in close proximity to the electrodes 1100. The electrodes 1100 can be installed on the test object using different methods, depending on the requirements of the specific application. In one embodiment, the electrodes 1100 are welded directly to the test object, providing a permanent and low-resistance electrical connection. Alternatively, for applications where welding is not desirable, the electrodes 1100 are machined to a sharp point and are spring-loaded against the test object. They can be held in place by a magnet 1400, strap 1400, or adhesive 1400, with the connection protected by a waterproof adhesive to prevent degradation. The electrodes 1100 are connected to the measuring instrument 1200 via a durable, low-resistance cable 1240. This cable 1240 is permanently attached to the electrodes 1100 at one end and features a standardized connector at the other end for easy connection to the measuring instrument 1200. The connection points are further protected by waterproof adhesive to ensure the long-term reliability of the system.

Referring now to FIG. 3, there is a diagram view of the embodiment of the system for non-destructive resistance monitoring 1000. In one exemplary use of the system 1000, the electrode pairs 1100A, 1100B are installed on a section of piping 2000 within a chemical processing plant. The electrodes 1100 are welded at predetermined intervals along the piping 2000, and the measuring instrument 1200 is installed in a location that is accessible for maintenance personnel. Once installed, the system 1000 continuously monitors the resistance between electrode pairs 1100A, 1100B, with data transmitted to a central monitoring station. Temperature sensors 1230 provide ambient temperature data, which is used to adjust the resistance measurements for accurate corrosion detection. Over time, the system 1000 identifies trends in the resistance data, enabling the early detection of corrosion and the proactive management of maintenance schedules.

In the shown embodiment, the two monitoring measuring instruments 1200 are disposed at a desired distance from each other (see FIG. 2) and include the same elements within the housings 1300, 1300A. The distance between the housings 1300, 1300A, as well as the characteristics of the pipe 2000 that extends therebetween, are known and variables used during the analysis. Each measuring instrument 1200 is equipped with a microcontroller unit (MCU) 1250, a data storage module 1260, and communication transceiver 1270, which facilitate the processing of resistance measurements, the storage of collected data, and the transmission of this data to the central monitoring station. The communication between the measuring instruments 1200 and the central monitoring station can be established via wired 1210 or wireless networks 1210, depending on the infrastructure of the chemical processing plant and the specific requirements for data security and transmission speed. Wireless configurations may utilize standard protocols such as Wi-Fi, Bluetooth, or cellular networks, offering flexibility in installation and scalability of the monitoring system. Wired connections, on the other hand, provide a reliable and secure means of data transmission, ideal for environments with high electromagnetic interference or stringent security policies. The inclusion of temperature sensors 1230 within or adjacent to the measuring instrument housing 1300 allows for real-time temperature compensation of resistance measurements, ensuring that the data reflects true material conditions unaffected by ambient temperature fluctuations. This configuration enables corrosion monitoring of critical infrastructure with minimal human intervention, significantly reducing the risk of undetected corrosion and its associated impacts on safety and operational efficiency.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A system for non-destructive resistance monitoring, comprising:
one or more pairs of electrodes, wherein each pair of electrodes include a first electrode for application of electrical current and a second electrode for measurement of potential in close proximity to the first electrode;
a measuring instrument coupled to the one or more pairs of electrodes, the measuring instrument configured to receive an electrical signal via the electrodes and collect material resistance data.

2. The system for non-destructive resistance monitoring of claim 1, wherein a resistance of a test object is determined by using two pairs of electrodes, wherein a current is applied between a pair of the first electrodes and measuring the voltage drop between a pair of the second electrodes.

3. The system for non-destructive resistance monitoring of claim 1, further comprising:
a network connecting the multiple measuring instruments to a central data collection point;
wherein the material resistance data is uploaded to a database for storage.

4. The system for non-destructive resistance monitoring of claim 3, wherein the database is adapted to store the material resistance data and provide it to a processor across the network.

5. The system for non-destructive resistance monitoring of claim 4, wherein the processor is adapted to analysis a plurality of material resistance data and to allow a user to access and analyze this data on demand.

6. The system for non-destructive resistance monitoring of claim 5, wherein a temperature sensor detects temperature data, wherein the processor continuously monitors the temperature data and the material resistance data to provide for data resistance monitoring over time.

7. The system for non-destructive resistance monitoring of claim 1, wherein each electrode is attached to the measuring instrument via a durable, low resistance cable.

8. The system for non-destructive resistance monitoring of claim 7, wherein the cable is permanently attached to the electrodes on one end (electrode end) and has a standardized connector to the measuring instrument at the other end.

9. The system for non-destructive resistance monitoring of claim 8, wherein the electrode end is coupled to the electrodes using a waterproof adhesive to protect the connection.

10. The system for non-destructive resistance monitoring of claim 8, wherein the measuring instrument is installed within a waterproof housing and the cables from the electrodes are attached within the waterproof housing.

11. The system for non-destructive resistance monitoring of claim 8, wherein electrodes are made of corrosion resistant stainless steel and are welded directly to the test object to provide a permanent test point.

12. The system for non-destructive resistance monitoring of claim 1, wherein electrodes are made of corrosion resistant stainless steel and are welded directly to the test object to provide a permanent test point.

13. The system for non-destructive resistance monitoring of claim 1, wherein hardened stainless steel electrodes which are machined to a point are spring loaded against the test object and held in place by a magnet, strap or adhesive.

14. The system for non-destructive resistance monitoring of claim 1, wherein the attachment mechanism and electrodes are then potted to the test object with a waterproof adhesive to protect the connection.
